(19) United States
(12) Patent Application Publication
(10) Pub. No.: US 2013/0280784 A1
(43) Pub. Date: Oct. 24, 2013

(54) ESCHERICHIA COLI EXPRESSION SYSTEM FOR PRODUCING MATURE HUMAN TYROSINASE AND A PRODUCING METHOD THEREOF

(71) Applicants: Shann-Tzong JIANG, Taoyuan County (TW); Gen-Hung CHEN, Taichung City (TW)

(72) Inventors: Shann-Tzong JIANG, Taoyuan County (TW); Gen-Hung CHEN, Taichung City (TW)

(73) Assignee: PROVIDENCE UNIVERSITY, TAICHUNG (TW)

(21) Appl. No.: 13/615,612

(22) Filed: Sep. 14, 2012

(30) Foreign Application Priority Data
Apr. 18, 2012 (TW) 101113789

(51) Int. Cl. C12N 9/00

(52) U.S. Cl. CPC C12N 9/0071
USPC 435/189; 435/252.33

(57) ABSTRACT

An E. coli expression system for producing mature human tyrosinase is provided and includes an E. coli host, which has a trait for expressing endogenous methionyl aminopeptidase in cytoplasm, and an expression vector transformed into the E. coli host. The expression vector has a replication origin sequence of an E. coli, and the expression vector includes an inducible promoter and a DNA fragment of human tyrosinase having a sequence referenced as SEQ ID NO:3, wherein a 5′ end of the DNA fragment is constructed at a restriction enzyme NdeI recognition site located at the downstream of the inducible promoter.

### RELATED APPLICATIONS

The application claims priority to Taiwan Application Serial Number 101113789, filed Apr. 18, 2012, which is herein incorporated by reference.

### SEQUENCE LISTING

The sequence listing submitted via EFS, in compliance with 37 CFR §1.52(e)(5), is incorporated herein by reference. The sequence listing text file submitted via EFS contains the file “TWT02292US-rsequencelisting”, created on Nov. 1, 2012, which is 2,997 bytes in size.

### BACKGROUND

1. Field of Invention

The present disclosure relates to a mass production method of mature human tyrosinase. More particularly, the present disclosure relates to a mass production method of recombinant mature human tyrosinase which utilizes an Escherichia (E. coli) expression system applying endogenous methionyl aminopeptidase of E. coli.

2. Description of Related Art

Tyrosinase, an oxidoreductase containing copper ions, is widely used in various fields such as cosmetics, food, medicine, the environment and so on.

The main function of tyrosinase is to oxidize polyphenol compounds. More importantly, tyrosinase plays a key role as a rate-limiting enzyme in a pathway, namely melanogenesis. The oxidation of tyrosine can be catalyzed by tyrosinase, so that pigments such as melanin can be produced. Therefore, much research has been carried out in recent times to discover inhibitors of tyrosinase so to block the production of such pigments.

Tyrosinase provided in tyrosinase inhibitory experiments is usually isolated from Agaricus bisporus and has been applied to mouse melanoma cells for studying experimental melanogenesis. Some studies, however, have shown that the biochemical characteristics and physiological activities of tyrosinase among species are lightly different.

Taking α-arbutin as an example, α-arbutin is a tyrosinase inhibitor which significantly inhibits tyrosinase of B16 mouse melanoma cells. Surprisingly, such a tyrosinase inhibitor, however, does not exhibit any inhibitory effects on tyrosinase isolated from Agaricus bisporus. This indicates that although both the aforementioned tyrosinases are isozymes, a plurality of characteristics thereof are significantly different so that the results of experiments designed for estimating tyrosinase activities regardless of whether using fungi or mammalian systems have been doubted.

In order to overcome the problem described above, some estimating methods in labs have been developed. For instance, human melanoma cells are used for experimental determinations in a wide range, and tyrosinase thereof has been directly extracted to be an enzyme source. Additionally, polymerase chain reaction (PCR) and western blotting are also commonly used for determining specific proteins in melanoma cells such as tyrosinase, tyrosinase related proteins (TRP1 and TRP2), microphthalmia transcription factor (MiTF) and melanocortin receptor 1 (MC1R), etc. Although the methods described above can be used to directly estimate the inhibitory effect on human melanoma cells, such methods are all extremely expensive.

### SUMMARY

An aspect of the present invention is to provide a cost-effective E. coli expression system and a tyrosinase producing method using the E. coli expression system that are not only used for mass producing mature human tyrosinase directly with significant efficiencies, but also may be used in place of conventional experimental tyrosinase isolated from Agaricus bisporus. In addition, the present invention can be widely used for discovering various human tyrosinase inhibitors.

According to one embodiment of the present disclosure, an E. coli expression system for producing mature human tyrosinase comprises an E. coli host and an expression vector. The E. coli host has a trait for expressing endogenous methionyl aminopeptidase in cytoplasm and the expression vector is transformed into the E. coli host. The expression vector has a replication origin sequence of an E. coli and includes an inducible promoter and a DNA fragment of human tyrosinase having a sequence referenced as SEQ ID NO:3. A 5′ end of the DNA fragment is constructed at a restriction enzyme NdeI recognition site located at the downstream of the inducible promoter.

According to another embodiment of the present disclosure, a method for producing mature human tyrosinase comprises the following steps of culturing an E. coli transformant in a liquid broth, inducing the inducible promoter during a mid-log phase of the E. coli transformant, overexpressing recombinant proteins as inclusion bodies, and refolding the recombinant proteins into a mature human tyrosinase in an active form. The E. coli transformant to be cultured has an expression system as the previous embodiment described. A temperature of the inducing step is not lower than 30° C. which the E. coli transformant can normally grow. The inclusion bodies of the recombinant proteins in the overexpressing step will be hydrolyzed in the cytoplasm of the E. coli transformant by an endogenous methionyl aminopeptidase to form a denatured mature human tyrosinase before the refolding step.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a schematic view illustrating a DNA fragment (SEQ ID NO:3) to be amplified with specific primers having recognition sequences of NdeI and XhoI, respectively, which are referenced as SEQ ID NO:1 and SEQ ID NO:2 according to one embodiment of the present disclosure;

FIG. 2 illustrates an expression vector of pET-23a(+)-RHT according to one embodiment of the present disclosure; and

FIG. 3 illustrates time course induction analysis of BL21(DE3)/pEP-23a(+)-RHT using SOS-PAGE analysis according to one embodiment of the present disclosure.

### DETAILED DESCRIPTION

In Prokaryotes, the first amino acid from the N-terminal of a newly synthesized protein, namely N-Formylmethionine (fMet), can be excised by a methionyl aminopeptidase while the second amino acid from the N-terminal has a short side chain, such as alanine, glycine, proline, threonine or valine. This process is called N-terminal methionine excision.

One embodiment of an E. coli expression system of the present disclosure involves processing a mass production of a mature human tyrosinase containing a sequence of 512 amino acids without any chemical modifications such as phosphorylation and glycosylation. This is because an endogenous methionyl aminopeptidase of E. coli can specifically hydrolyze the peptide bond formed between Methionine and Glycine (Met-Gly). In addition, the codon GGC in position 58 to 60 of the human tyrosinase gene is encoded as glycine, and a DNA fragment designed for representing the human tyrosinase gene is constructed into an expression vector having a recognition sequence (CATATG) of a restriction enzyme NdeI incorporated at the 5′ end, thus forming recombinant proteins containing a fragment of fMet-Gly at the 5′ end of the DNA fragment. Therefore, the recombinant protein can be hydrolyzed by methionyl aminopeptidase spontaneously after translation.

Furthermore, to effectively obtain mass mature human tyrosinase, the aforementioned embodiment of the present disclosure has chosen an E. coli host and an inducible promoter for producing proteins with high efficiencies. Therefore, during protein production using the high efficiency E. coli host and inducible promoter mentioned above, proteins will be over-expressed and aggregated, thus forming inclusion bodies. The inclusion bodies can be solubilized, renatured and refolded to form proteins having normal biological activities by changing buffer conditions such as the concentration of salts or pH values, and such processes can be performed by anyone skilled in the art.

When the expression of the inserted genes have been induced and the corresponding proteins start to be over-expressed in a host, major resources of cells are occupied for processing mass productions of recombinant proteins of the insert genes. Therefore, although proteins forming inclusion bodies are insoluble and do not have the normal biological functions, inclusion bodies still provide much higher concentrations and a greater purity of recombinant proteins compared with other soluble endogenous proteins in cells. Thus, mass producing recombinant proteins by using the over-expressing process is highly cost-effective.

### Examples

The following examples are described for those skilled in the art to further understand the present disclosure and should not be limited to the present disclosure.

I. Cloning of Recombinant Human Tyrosinase E. coli Transformant

A method for producing E. coli transformant by using an expression vector pET-23a(+)-RHT having constructed recombinant human tyrosinase is described as an example to introduce an E. coli expression system of the present disclosure.

The DNA fragment to be constructed into an expression vector for expressing human tyrosinase was obtained by using total cDNA, which was reverse transcribed from the total RNA of human melanoma, as a template to specifically amplify the cDNA of human tyrosinase with specific primers referenced as SEQ ID NO:1 and SEQ ID NO:2. In addition, specific primers referenced as SEQ ID NO:1 and SEQ ID NO:2 were also designed to incorporate recognition sequences of restriction enzymes, NdeI and XhoI, to the 5′ ends of both strands of the human tyrosinase cDNA, respectively. As a result, the DNA fragment contains 1551 base pairs, and the total sequence the DNA fragment is referenced as SEQ ID NO:3.

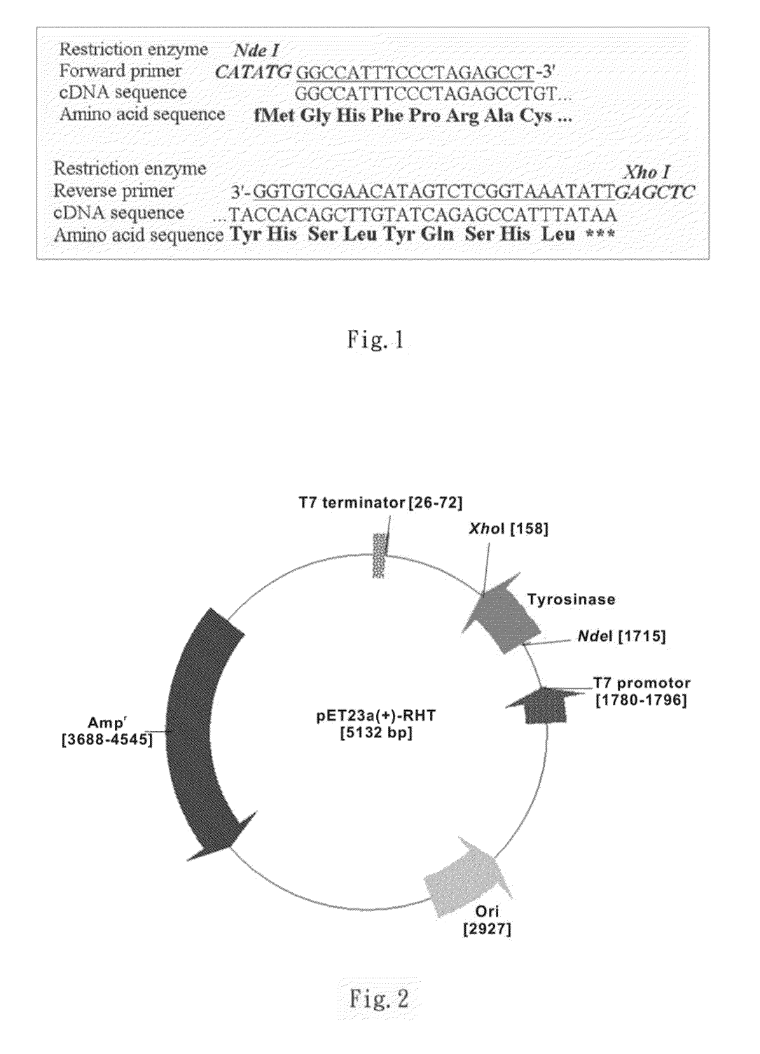

FIG. 1 is a schematic view illustrating the DNA fragment (SEQ ID NO:3) to be amplified with specific primers having recognition sequences of NdeI and XhoI, respectively, which are referenced as SEQ ID NO:1 and SEQ ID NO:2 according to one embodiment of the present disclosure. The DNA fragment to be amplified was designed to have a corresponding amino acid sequence starting with a formylmethionine at the N-terminus followed by a glycine as the second amino acid in the sequence after translation. The peptide bond between fMet-Gly can be spontaneously hydrolyzed by an endogenous enzyme called methionyl aminopeptidase in E. coli. Additionally, the most important thing is that the 20^{th} to the 531^{st} amino acids of an amino acid sequence representing mature human tyrosinase vas retained after the spontaneous cleavage of the peptide bond between fMet-Gly by the endogenous methionyl aminopeptidase in E. coli cytoplasm.

Human melanoma was cultured in a liquid medium containing 2 mM of L-glutamine, 1.5 g/L of sodium bicarbonate, 0.1 mM non-essential amino acids, 1.0 mM sodium pyruvate and 10% of fetal bovine serum under a condition of 37° C. and with 5% carbon dioxide. After the cells matured, a TRIzol® reagent was applied for extracting the total RNA of the cells. These extracted total RNAs were then reverse transcribed into cDNA by a reverse transcriptase (SuperScript RT II GIBC0 BRL).

Sequences of the specific primers referenced as SEQ ID NO:1 and SEQ ID NO:2 for amplifying human tyrosinase cDNA were designed by referring to the genome database of National Center for Biotechnology Information (NCBI). By using a polymerase chain reaction, the recognition sequences of restriction enzymes, NdeI and XhoI can be incorporated to the 5′ ends of both strands of the human tyrosinase cDNA, respectively, to form DNA fragments which contain 1551 base pairs. The total sequence the DNA fragments is referenced as SEQ ID NO:3. Plus, such DNA fragments can be amplified by using a polymerase chain reaction as well.

In greater detail, the polymerase chain reaction (PCR) mentioned above was performed using 25 pg of human tyrosinese cDNA template, 5 μL of 10×PCR buffer (Pfx50™), 3 mM of deoxynucleotide triphosphate, 80 μM of primer referenced as SEQ ID NO:180 μM of primer referenced as SEQ ID NO:25 units of DNA polymerase (Pfx50™), and deionized water for adjusting the final volume to 50 μL for the following PCR cycles. One PCR cycle of predenaturation (94° C. for 2 min) followed by 25 PCR cycles in an order of denaturation (94° C. for 30 s), annealing (56° C. for 30 s), and extension (68° C. for 90 s); and one PCR cycle of final primer extension (68° C. for 10 min). Afterwards, DNA fragments amplified by using a polymerase chain reaction was further analyzed utilizing 1% agarose gel electrophoresis analysis.

The DNA fragment (SEQ ID NO:3) contains restriction sites of NdeI and XhoI at the 5′ ends of both strands thereof, respectively. Also, the pET-23a(+) expression vector was sequentially cleaved by the two aforementioned restriction enzymes. Subsequently, the DNA fragment (SEQ ID NO:3) can be constructed into the pET-23a(+) expression vector by a T₄ DNA ligase, and the whole constructed expression vector (pET-23a(+)-RHT) can be transformed into E. coli Top 10 F′ competent cells. Afterwards, the transformed cells were plated on Luria-Bertani (LB) agar plates containing Ampicillin to select transformants containing expression vectors of pET-23a(+)-RHT (total 5132 base pairs). The constructed expression vector of pET-23a(+)-RHT is shown in FIG. 2.

The constructed expression vector of pET-23a(+)-RHT transformed into E. coli/BL21(DE3) for induction and expression is represented BL21(DE3)/pET-23a(+)-RHT in the following descriptions.

### II. Expression of Mature Human Tyrosinase in E. Coli

### 1. Induction Temperature for Producing Recombinant Mature Human Tyrosinase

The E. coli BL21(DE3) transformed with pET-23a(+)-RHT constructed expression vector was cultured in an LB broth containing Ampicillin at 37° C. and for 10-12 hours using a shaking incubator (150 rpm), namely the activated culture. The activated culture was subcultured into twenty times the volume of the activated culture of fresh LB broth containing 100 μg/mL Ampicillin and 10 μM CuSO4 in a flask. When the absorbance at an optical density of 600 nm (O.D.₆₀₀) reached 0.6, that is, cells were grown to mid-log phase, IPTG was added to a final concentration of 0.1 mM, and the shaking continued for cultivation at 30 to 37° C. and for 3-12 hours to induce the expression of RHT protein.

In general, people skilled in the art usually focus on how to reduce the formation of inclusion bodies using an E. coli expression system. For example, culturing an E. coli expression system at an induction temperature lower than 30° C. is more likely to obtain soluble recombinant proteins by decreasing the expression efficiency, and this is because the recombinant proteins might have longer durations to fold into their native forms. On the other hand, in the present disclosure, a relatively high temperature (37° C.) was chosen to be the induction temperature for producing recombinant proteins with higher efficiencies, thus obtaining recombinant proteins formed as inclusion bodies.

Cells were harvested by centrifuging the cultured fluid after induction at 2,500×g for 10 minutes and were resuspended with a buffer A (50 mM Tris-HCl, 50 mM NaCl, pH 7.5). Cells were then sonicated and lysed by a sonicator (output power: 240 W, XL-2020 SONICATOR) in an ice-bath for 30 minutes with cycles of one short burst of 10 seconds followed by intervals of 5 seconds for cooling. Finally, the supernatant and the cell debris were collected by centrifugation at 9,000×g and 4° C. for 10 minutes.

### 2. Induction Time During the Expression of Recombinant Mature Human Tyrosinase

In order to obtain mass produced mature human tyrosinase efficiently, a time course induction analysis of BL21(DE3)/pEP-23a(+)-RHT using SDS-PAGE analysis was provided for interpreting the process for inducing the expression of mature human tyrosinase of an example of the present disclosure.

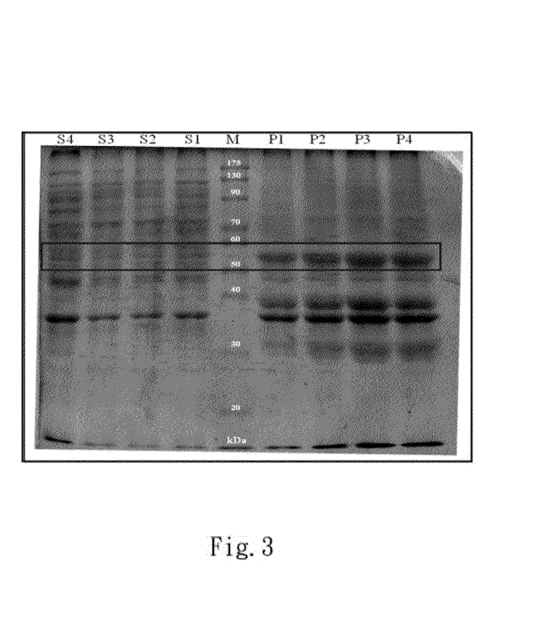

FIG. 3 illustrates a time course induction analysis BL21(DE3)/pEP-2a(+)-RHT using SDS-PAGE analysis. Lane S1, S2, S3 and S4 represent proteins of the soluble portion of BL21(DE3)/pEP-23a(+)-RHT after induction durations of 3, 6, 9 and 12 hours, respectively. Lane of P1, P2, P3, and P4 represent proteins of the insoluble portion of BL21(DE3)/pEP-23a(+)-RHT after induction durations of 3, 6, 9 and 12 hours, respectively. Lane M represents a molecular weight ladder of a low molecular weight protein marker.

As shown in FIG. 3, recombinant human tyrosinase (including 512 amino acids, and the molecular weight thereof is about 57 kDa) over-expressed from the E. coli expression system of the present disclosure were majorly retained in the insoluble portion of lysed BL21(DE3)/pEP-23a(+)-RHT. Proteins expressed at 37° C. with an induction duration of 3 hours have started to aggregate and thus form inclusion bodies. A significantly greater amount of recombinant human tyrosinase forming inclusion bodies can be obtained by expressing proteins at 37° C. with longer induction durations (more than 9 hours).

### 3. Renaturation of the Recombinant Human Tyrosinase

Inclusion bodies of recombinant human tyrosinase of the present disclosure, obtained by expressing BL21(DE3)/pEP-23a(+)-RHT at 37° C. with an induction duration of 9 hours, contain 45% hydrophobic amino acids, and further include 17 Cysteine residues.

A renaturation procedure for renaturing aggregated recombinant human tyrosinase which forms inclusion bodies is disclosed herein. In order to collect the lysed cells after centrifugation, buffer A (about 9 times of the volume of the lysed cells) was added to the lysed cells and mixed homogeneously. Subsequently, after standing for 5 minutes, the mixture was centrifuged at 9000×g and 4° C. for 10 minutes. Supernatant was then removed and the above steps were repeated three times. Next, 1-4% Sodium dodecyl sulfate (SOS), 0.5-3% 2-mercaptoethanol, 7% Glycerol and 25 mM Tris-HCl with a pH value of 7.5 were added to the pellet and this mixture was left to react for 4 hours at room temperature. The whole mixture was loaded into an activated dialysis membrane (Spectra/Por® Membrane MWCO: 3,500) and the whole mixture was dialyzed against buffer B (0.5% Triton X-100, 25 mM Tris-HCl, pH 7.5) for 4 hours. Afterwards, buffer B was substituted with buffer C (25 mM Tris-HCl, 10 mM CuSO4, pH 7.5) and proteins were refolded for another 4 hours. Used buffer C was then substituted with fresh buffer C and again protein refolding was continued for another 4 hours, and this step (substitution of used and fresh buffer C) was repeated three times. The dialysis membrane was then removed from buffer C carefully, and then the containing mixture was centrifuged for discarding the retained unfolded, inclusion bodies. The soluble portion, that is, the crude extract of refolded recombinant human tyrosinase can be collected.

In order to analyze the enzyme activity of the aforementioned refolded recombinant human tyrosinase, 400 ml of 50 mM PBS buffer (pH 7.0), and 300 ml crude extract containing refolded recombinant human tyrosinase were mixed and left standing for 5 minutes at 37° C. Subsequently, 300 ml L-Dopa was added (to a final concentration of 3 mM) to the mixture as an enzyme substrate and reacted for 30 minutes at 37° C. The absorbance at A₄₇₅ was measured using a spectrophotometer.

Referring to the result, the enzyme substrate L-Dopa can be oxidized and the color thereof turns black, thus proving the crude extract has tyrosinase activity.

In summary, the peptide bond between Methionine and Glycine of the recombinants proteins expressed using the E. coli expression system of the present disclosure can be specifically recognized and cleaved by the endogenous methionyl aminopeptidase of E. coli, thus removing the redundant formylmethionine at the N-terminus. Human mature tyrosinase containing 512 amino acids can then be obtained, and its normal biological activity can be recovered after renaturation and refolding processes

### 4. Purification of Recombinant Human Tyrosinase

As described above, the rude extract of recombinant human tyrosinase has tyrosinase activity after renaturation and refolding. Such a crude extract can be further purified to isolate the pure recombinant human mature tyrosinase.

The conditions and tools used in the purification method disclosed in the present disclosure can be modified by persons having ordinary skill in the art. Thus, the example described below is not used for limiting the scope of the claims of the present disclosure.

First of all, the aforementioned crude extract is concentrated using Amicon ultra centrifugal filter units (YM 3, cutoff: 3,000) at 4° C. with nitrogen. Subsequently, the condensed crude extract was separated using gel filtration chromatography with a Sephacryl S-100 HR column (1.6×100 cm). 0.5 ml of condensed crude extract was injected into the column, which has been pre-equilibrated using 25 mM Tris-HCL buffer (pH 7.5). The condensed crude extract was then separated against 25 mM Tris-HCl buffer (pH 7.5) with a flow rate of 0.5 ml/min. During separation, the UV absorbance of the effluent was monitored by a UV detector (Pharmacia US-I) at A₂₈₀. Fractions of 2 ml ere collected and analyzed for tyrosinase activity.

Afterwards, fractions which mainly contain recombinant human tyrosinase were loaded onto a DEAE sepharose Fast Flow column (2.6×10 cm) to process Ion-Exchange chromatography for a secondary purification. In detail, a 10 ml mixture of the fractions which mainly contain recombinant human tyrosinase were injected into the column, which has been pre-equilibrated using 25 mM Tris-HCL buffer (pH 7.5). The mixture was then separated against 25 mM Tris-HCl buffer (pH 7.5) with a flow rate of 0.5 ml/min. During separation, the UV absorbance of the effluent was monitored by a UV detector (Pharmacia US-I) at A₂₈₀. After the UV absorbance of the effluent monitored by a UV detector (Pharmacia US-I) was stabilized, a gradient from 0M to 1M of sodium chloride was applied to the column for eluting proteins retaining on the column. Fractions of 2 ml were collected and analyzed for tyrosinase activity.

Furthermore, hydrophobic-interaction chromatography using Macro-Prep® HIC support column (2.6×10 cm) may be used as another option for isolating recombinant human tyrosinase from the crude extract of recombinant human tyrosinase. The crude extract of recombinant human tyrosinase was slowly added to a buffer containing 25 mM Tris-HCl and 0.2 M NaCl, pH 7.5, with an equal volume (crude extract:buffer=1:1). The mixture was injected into the column, which has been pre-equilibrated using 25 mM Tris-HCl buffer with 0.1 M NaCl (pH 7.5). The mixture was then separated with a flow rate of 0.5 ml/min. During separation, the UV absorbance of the effluent was monitored by a UV detector (Pharmacia US-I) at A₂₈₀. After the UV absorbance of the effluent monitored by a UV detector (Pharmacia US-I) was stabilized a 25 mM Tris-HCl buffer was applied to the column for eluting proteins retaining on the column. Fractions of 2 ml were collected and analyzed for tyrosinase activity.

The final yield of the purification, the total amount of the proteins, the enzyme activity and the analysis of the specificity were summarized in Table 1. Table 1 summarizes the results of 10 L of the starting material, and L-Dopa was used as an enzyme substrate in the enzyme activity assay.

TABLE 1
Analysis of the crude extract produced by the E. coli expression system

| | Total | Total | | | |
|---|---|---|---|---|---|
| | enzyme | protein | | | |
| Purification | activity | amount | Specificity | Yield | Purity |
| state | (units) | (mg) | (units/mg) | (%) | (times) |
| Crude extract | 3457.8 | 540.0 | 6.4 | 100.0 | 1.0 |
| DEAE ion-exchange | 1383.1 | 89.0 | 15.5 | 40.0 | 2.4 |
| Gel filtration | 144.3 | 2.5 | 57.7 | 4.2 | 9.0 |

As shown in Table 1, the crude extract containing the recombinant human tyrosinase exhibits tyrosinase activity (6.4 units 1 mg). After isolation of recombinant human tyrosinase from the crude extract using ion-exchange chromatography, the tyrosinase activity can reach 15.5 units/mg, whereas the tyrosinase activity can even reach 57.7 units 1 mg by using gel filtration chromatography.

In short, advantages of producing recombinant human tyrosinase by using the E. coli expression system of the present disclosure are described as follows:

1. The expression vector transformed into the E. coli expression system of the present disclosure is designed to simply use endogenous methionyl aminopeptidase for spontaneously removing the first amino acid residue, formylmethionine, after translation and thus simplifying the expressing steps compared with the conventional recombinant protein expression methods (conventionally, post-translational modifications must be manipulated artificially).

2. The example of the present disclosure successfully produced tyrosinase having its normal biological activity. In addition, using human source tyrosinase can easily overcome the experimental disadvantages or errors aforementioned of using tyrosinase isolated from other species, like Agaricus bisporus, due to slight differences of biochemical characteristics and physiological activities of tyrosinase among various species.

3. The advantages of the E. coli expression system have been fully utilized in the present disclosure, such as high protein expression efficiency, ease of manipulating various conditions, ease of operation, and so on. Therefore, the present invention not only provides a protein producing method with normal biological activities, but largely decreases the cost of producing enzymes, and thus satisfies various the requirements of any manufacturer.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the invention. In view of the foregoing, it is intended that the present disclosure cover modifications and variations of this invention provided they fall within the scope of the following claims.

What is claimed is:

1. An E. coli expression system for producing mature human tyrosinase, comprising:
an E. coli host having a trait for expressing endogenous methionyl aminopeptidase in cytoplasm; and
an expression vector transformed into the E. coli host, wherein the expression vector has a replication origin sequence of an E. coli, and the expression vector includes:
an inducible promoter; and
a DNA fragment of human tyrosinase having a sequence referenced as SEQ ID NO:3, wherein a 5′ end of the DNA fragment is constructed at a restriction enzyme NdeI recognition site located at the downstream of the inducible promoter.

2. The expression system of claim 1, wherein the E. coli host is a BL21(DE3) strain, and the inducible promoter is a T7 promoter.

3. The expression system of claim 2, wherein the expression vector originates from a pET23a(+) plasmid, and a 3′ end of the DNA fragment is constructed at a restriction enzyme XhoI recognition site located in the downstream of the inducible promoter.

4. A method for producing mature human tyrosinase, comprising:
culturing an E. coli transformant in a liquid broth, wherein the E. coli transformant has an expression system of claim 1;
inducing the inducible promoter during a mid-log phase of the E. coli transformant at a temperature of not lower than 30° C. which the E. coli transformant can normally grow;
overexpressing recombinant proteins as inclusion bodies, wherein the recombinant proteins will then be hydrolyzed in cytoplasm of the E. coli transformant by an endogenous methionyl aminopeptidase to form a denatured mature human tyrosinase; and
refolding the recombinant proteins into a mature human tyrosinase in an active form.

5. The method of claim 4, wherein the E. coli transformant is a BL21(DE3) strain, and the E. coli transformant comprises an expression vector having a T7 promoter.

6. The method of claim 5, wherein the inducing step takes 3 to 12 hours.

7. The method of claim 5, wherein the inducing step takes 9 hours.

8. The method of claim 5, wherein the temperature of the inducing step is not lower than 37° C.

9. The method of claim 5, wherein the refolding step comprises:
dissolving the inclusion bodies in a solubilization buffer containing 1% of sodium lauryl sulfate and 0.5% of 2-mercaptoethanol; and
refolding the inclusion bodies in a buffer containing copper ions.

10. The method of claim 9, further comprising:
purifying the recombinant proteins by gel filtration chromatography after the refolding step of the recombinant proteins.


SEQUENCE LISTING (3 sequences)

SEQ ID NO: 1 - DNA, 28 bp, Artificial Sequence
  Forward primer for amplifying human tyrosinase gene using PCR
atacatatgggccatttccctagagcct

SEQ ID NO: 2 - DNA, 37 bp, Artificial Sequence
  Reversed primer for amplifying human tyrosinase gene using PCR
atactcgagttataaatggctctgatacaagctgtgg

SEQ ID NO: 3 - DNA, 1551 bp, Artificial Sequence
  DNA fragment for transcription of human tyrosinase
catatgggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatccattattatgtgtcaatggatgcactgcttgggggatatgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataactcgag